(19) United States
(12) Patent Application Publication
(10) Pub. No.: US 2008/0149101 A1
(43) Pub. Date: Jun. 26, 2008

(54) Therapeutic arrangement

(76) Inventors: Uwe Becker, Eichenau (DE); Jochim Koch, Ratzeburg (DE); Bernhard Lorenz, Marzling (DE)

Correspondence Address: WALTER OTTESEN, PO BOX 4026, GAITHERSBURG, MD, 20885-4026, US

(21) Appl. No.: 12/000,681

(22) Filed: Dec. 17, 2007

(30) Foreign Application Priority Data
Dec. 15, 2006 (DE) 10 2006 059 340.5

(51) Int. Cl. A61M 16/10; A62B 7/00; A62B 9/00

(52) U.S. Cl. 128/204.17; 128/200.24; 128/204.21

(57) ABSTRACT

A therapeutic apparatus for medical applications is improved in such a manner that the capacity of an energy store can be utilized as best as possible when there is a breakdown of the power supply. A charge level detector (16) is provided for the energy store which reduces the power output of the energy consumer (6) when there is a falling charge level of the energy store (14) with a drop of the supply voltage (15).

### CROSS REFERENCE TO RELATED APPLICATION

This application claims priority of German patent application no. 10 2006 059 340.5, filed Dec. 15, 2006, the entire content of which is incorporated herein by reference.

### FIELD OF THE INVENTION

The invention relates to a therapeutic arrangement for applications in medicine.

### BACKGROUND OF THE INVENTION

U.S. Pat. No. 5,950,621 discloses a ventilating apparatus wherein ambient air is drawn by suction through a filter by means of a pump unit and is supplied to a patient with a breathing mask. The pump unit comprises a blower which is connected to a battery via a data collection unit.

The data collection unit monitors the voltage of the battery and warns the user of impending battery failure via optical and acoustic alarm devices. The alarm indication can be outputted as a simple signal or as a complex warning sequence. In addition, blinking lights, intensity modulations or color changes can be used in order to indicate different stages of the alarm. Furthermore, a power supply apparatus is provided in order to buffer or recharge the battery.

The disadvantage of the known arrangement is that only the remaining residual capacity of the battery is indicated and the user can only estimate how long the ventilation apparatus is still operationally ready. If no power mains is available, then a complete failure of the gas supply occurs when the battery is completely exhausted.

### SUMMARY OF THE INVENTION

It is an object of the invention to improve an arrangement of the kind described above so that the capacity of an energy store supplying the arrangement is utilized in the best possible manner. It is also an object of the invention to provide a method for operating the arrangement.

The arrangement of the invention is for medical applications. The arrangement includes: a consumer of energy; an energy source for operating the consumer; the energy source including an energy store having a charge level which changes when energy is drawn therefrom; a detector for detecting the charge level; and, means for reducing the power output/power input of the consumer when the charge level detected by the detector drops off.

The advantage of the invention is essentially that, with an impending exhaustion of the energy store, not only is a warning announcement outputted but the power output of the energy consumer is reduced in order to utilize the remaining energy supply over the longest possible time span. With a medical apparatus, functions necessary for life have the highest priority and are not permitted to be reduced whereas reductions are possible with respect to functions related to comfort.

According to a feature of the invention, the power input/power output of the energy consumer is adapted to the energy supply of the energy store. This adaptation can be so undertaken that the power input/power output is correspondingly tracked when there is a falling energy supply in order to achieve the longest possible use time of the therapeutic arrangement. The adaptation can be carried out proportionally to the decrease of the energy supply or can be undertaken based on a pregiven characteristic line. In the context of the invention, different characteristic lines can be held ready which, on the one hand, are adapted to the energy consumer and, on the other hand, contain settings specific to the user.

Heat sources in humidifiers or radiation heaters and mattress heaters in thermotherapeutic apparatus, for example, are energy consumers.

During in-hospital treatment in a medical treatment room, there is normally always a mains supply present in addition to an energy store. Accordingly, for example, the processor unit of a humidifier, which carries out the control of the heat source, makes routine inquiries as to the presence of a mains supply. The charging level of the energy store is continuously monitored with a charge level detector. If there is an interruption of the network power supply, then a switchover to the energy store takes place immediately and, depending upon the charge level of the energy store, an operating mode is selected by the processor unit with which the humidifier can be operated as long as possible.

In a first operating mode, it is provided by way of example that the full power output is available when there is a charge level of the energy store of greater than 75%. If the charge level lies between 50% and 75%, the power output of the heat source is reduced by 5% in a second operating mode. In a third operating mode, with a charge level between 10% and 50%, the power output is reduced by a further 10%. In contrast, if the charge level drops below 10% of the total capacity, the heating source is switched off.

It is advantageous when the user can himself or herself change the settings for reducing the energy. Accordingly, the user could impart to the system that a switchover into an operating mode should not take place because the user is sure that the interruption of the power source will not exceed a certain time duration. On the other hand, the user could just as well select, ab initio, the operating mode having the greatest energy savings because the power source will not be available for a presumably long time. One could also, in addition, conceive of a selection from differently structured profiles. One of these profiles is the simple power reduction corresponding to predetermined operating modes. A further profile could be directed to a maximum patient comfort; however, then a shorter running time is to be expected since the energy store is exhausted fastest. In this case, the power of the system would not be reduced or reduced only very slightly. An alternative profile could be designed for maximum running time without mains supply. In this case, reductions as to comfort for the patient are to be made because this results in a very substantial reduction of the capacity of the systems. In addition, it is conceivable that a certain number of additional profiles is provided which can be freely configured by the user.

For other apparatus, such as anesthesia apparatus, one can save energy in that the heater of the breathing system and/or the heater of the breathing gas hose is throttled. In a system having infusion pumps, the savings of energy is possible only for pumps which do not actually pump at the particular time. These pumps could be placed in an operationally ready mode or these pumps can be completely switched off.

Measuring apparatus, which do not instantaneously measure, can be also placed in the operationally ready mode. For some measuring parameters, which change only slightly, it can be advantageous to reduce the sampling rate or scanning rate. Accordingly, the measurement of the temperature is a procedure which delivers good results at a low sampling rate. One could therefore reduce the scanning rate, for example, from 100 Hertz to 10 Hertz without negatively influencing the patient's safety or quality of diagnosis. In addition, one could increase the interval of an automatic non-invasive blood pressure measurement. With a further parameter such as the pulse oximetry, one could transfer from the continuous measurement to a non-continuous measurement of the parameter. Accordingly, one could, for example, detect the value only once each second and switch the sensors so that they are non-conductive during the remaining time.

Transcutaneous gas measurements are likewise suitable for saving energy by changing the clock rate. Also, for transcutaneous measurements, one could reduce the temperature of the sensors. This would have the consequence of a reduction of the accuracy of the measured values but would be mostly acceptable with an appropriate confirmation especially with the background that the trend of the parameter is of greater interest than the absolute measured value.

In a complete workplace, components not currently needed can be transferred into the operationally ready mode for saving energy. If needed, however, a previous confirmation of the user has to be obtained.

A workstation for a neonatology ward comprises a thermal bed, a ventilating apparatus, infusion pumps, a patient monitoring system and a central control and display unit.

A workplace for an intensive care unit comprises one or several ventilating apparatus, infusion pumps, a patient monitoring system and a central control and display unit.

An anesthesia workplace comprises an anesthesia apparatus, infusion pumps, a patient monitoring system and a central control and display unit.

In a thermotherapeutic apparatus, a radiation heater can be throttled or switched off completely to save energy when there is a failure of the mains supply and only the mattress heater continues to be operated. In addition, the possibility is present to also throttle the mattress heater in a further step to save energy until the energy store is completely exhausted.

With an incubator, the breathing air for the patient is likewise humidified. Here, to save energy, one would first reduce the humidification or switch it off entirely. Only thereafter would a reduction of the temperature in the incubator take place to the extent that this tolerable.

The method of the invention is for operating an arrangement for medical applications which includes a consumer of energy and an energy store for operating the consumer, the energy store having a charge level which changes as energy is drawn therefrom. The method includes the steps of: monitoring the charge level of the energy store with a charge level detector; and, reducing the power output/power input of the consumer when the charge level drops off.

### BRIEF DESCRIPTION OF THE DRAWINGS

The invention will now be described with reference to the drawings wherein:

FIG. 1 is a schematic showing a humidifier according to an embodiment of the invention;

FIG. 2 is a schematic showing a thermotherapeutic apparatus according to the invention; and,

FIG. 3 is a schematic showing a measuring apparatus for making transcutaneous measurements of gases in the blood.

### DESCRIPTION OF THE PREFERRED EMBODIMENTS OF THE INVENTION

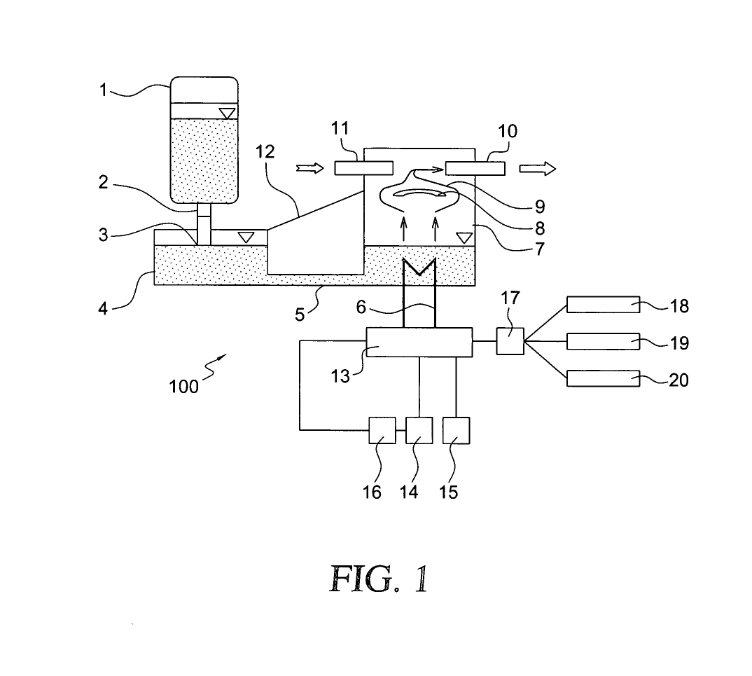

FIG. 1 shows the configuration of a humidifier 100 for breathing gas. Sterile water is held ready in an exchangeable, pressure-stable closed water supply vessel 1. The water supply vessel 1 is connected to an intermediate store 4 via a connector 2. In this way, water can run out of the water supply vessel 1 into the intermediate store 4 of the humidifier system until the water level has risen so far that the channel 3 of the connector 2 is closed and no air can any longer flow into the water supply vessel 1. The water is conducted into the vaporizer chamber 7 via a water connecting line 5 having a diameter of approximately 1 to 2.5 millimeters.

In the vaporizer chamber 7, the heater 6 ensures the necessary energy to heat up the water and to cause the water to boil and vaporize. The water vapor rises in the vaporizer chamber 7 and is redirected by a shield 8 so that water vapor reaches the inhalation gas. The water vapor is conducted along the path 9 into the outlet 10 and, from there, mixes with the inhalation gas to the patient which comes from the inlet 11. A pressure equalization between the outlet 10 and the intermediate store 4, which acts as a water level controller, is established via a gas pressure equalizing line 12.

The heater 6 is connected via a processor unit 13 to an energy store 14 and a power supply 15. A charge level detector 16 continuously detects the charge level of the energy store 14 and transmits corresponding data to the processor unit 13. Three operating modes (18, 19, 20) for operating the heater 6 can be selected by means of a selector switch 17 in dependence upon the charge level of the energy store 14.

In a first operating mode 18, the full power output is available for a charge level greater than 75%.

A second operating mode 19 provides that the power output of the heat source 6 is reduced by a first pregiven value of approximately 5% when the charge level is between 50% and 75%. In a third operating mode 20, the heating power is further reduced by a second pregiven value of approximately 10% when the charge level lies between 10% and 50%. The heat source 6 is switched off when the charge level drops below a third pregiven value of approximately 10% of the total capacity.

The processor unit 13 monitors whether the mains or central power supply 15 is present. If the mains supply 15 is present, then the energy store 14 is charged. If the mains supply 15 fails, then the charge level detector 16 transmits corresponding data to the processor unit 13 and an operating mode (18, 19, 20), which corresponds to the charge level, is selected with the selection switch 17.

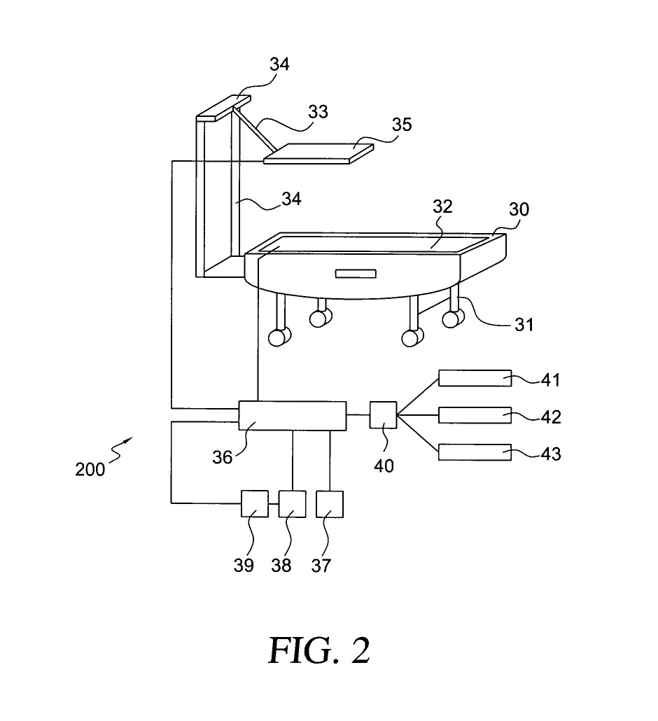

FIG. 2 shows a thermotherapeutic apparatus 200 for neonates which includes as essential components: a bed surface 30 mounted on an undercarriage 31; a mattress heater 32 on the bed surface 30; and, a radiation heater 35 which is pivotable about a joint 33 on a carrier arm 34. The mattress heater 32 and the radiation heater 35 are connected via a processor unit 36 to a mains supply 37 and an energy store 38. A charge level detector 39 continuously detects the charge level of the energy store 38 and transmits corresponding data to the processor unit 36.

The processor unit 36 monitors whether the power supply 37 is present. If the power supply 37 malfunctions, then there is a switchover to the energy store 38 and the charge level detector 39 transmits the instantaneous charge level to the processor unit 36.

With a selector switch 40, three operating modes (41, 42, 43) can be selected for the radiation heater 35. The mattress heater 32 continues to be operated at constant power.

In a first operating mode 41 with a charge level of the energy store 38 of greater than 75%, the full power output is made available to the radiation heater 35. In a second operating mode 42 with a charge level between 50% and 75%, the power output of the radiation heater is reduced by 5%. In a third operating mode 43 with a charge level between 20% and 50%, the power output of the radiation heater 35 is additionally reduced by 10%. The radiation heater 35 is switched off when the charge level of the energy store 38 drops below 20%.

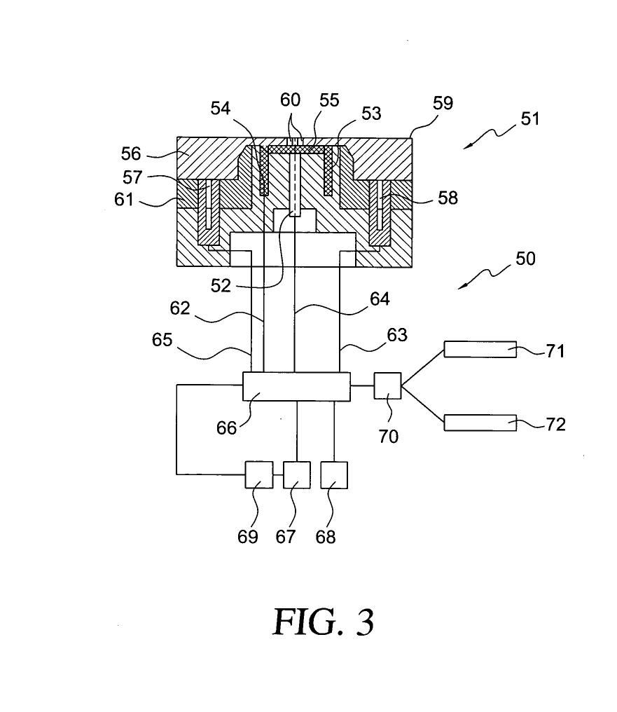

FIG. 3 shows a measurement value recorder 50 for transcutaneous measurement of gases in the blood. The measurement value recorder 50 includes an electrochemical measuring cell 51 having a measuring electrode 52, an electrolyte chamber 53 and a reference electrode 54. The electrolyte chamber 53 is covered by a membrane 55. An electrically operated heater disc 56 is disposed on a thermal insulating body 61. The heater disc 56 is held with insert contacts (57, 58) and is electrically contacted thereby. The heater disc 56 functions to hyperaemize the skin and lies with a surface 59 against the surface of the skin. The gas, which extravasates from the skin and is to be measured, reaches the measuring electrode 52 via apertures 60.

The electrochemical measuring cell 51 and the heater disc 56 are connected to a processor unit 66 via connecting lines (62, 63, 64, 65). The processor unit 66 evaluates the measurement signals of the electrochemical measuring cell 51 and heats the heater disc 56 to a predetermined temperature. This processor unit 66 is connected to an energy source 67 and a central power supply 68. A charge level detector 69 continuously detects the charge level of the energy store 67 and transmits corresponding data to the processor unit 66. Two operating modes (71, 72) can be set via a selector switch 70 actuated by the processor unit 66.

In a first operating mode 71, the full heater power for the heater disc 56 is available when there is a charge state of the energy store 67 of greater than 75%. In a second operating mode 72, the power output of the heater disc 56 is reduced by 20% below a charge level of 75%. In addition, in the second operating mode 72, the clock rate of the processor unit 66 is reduced which leads to an additional savings of energy. Since transcutaneous gas concentration measurement values change only slowly, the evaluation at a reduced clock rate is acceptable.

It is understood that the foregoing description is that of the preferred embodiments of the invention and that various changes and modifications may be made thereto without departing from the spirit and scope of the invention as defined in the appended claims.

What is claimed is:

1. An arrangement for medical applications, the arrangement comprising:
a consumer of energy;
an energy source for operating said consumer;
said energy source including an energy store having a charge level which changes when energy is drawn therefrom;
a detector for detecting said charge level; and,
means for reducing the power output/power input of said consumer when said charge level detected by said detector drops off.

2. The arrangement of claim 1, wherein said consumer is a radiant heater of a thermotherapeutic apparatus for open treatment.

3. The arrangement of claim 1, wherein said consumer is a heat source in a humidifier for imparting moisture and heat to a breathing gas.

4. The arrangement of claim 3, wherein said humidifier is mounted in an inhalation line of a ventilating apparatus.

5. The arrangement of claim 3, wherein said humidifier is disposed in an incubator for imparting moisture to air in the interior space of said incubator.

6. The arrangement of claim 1, wherein said consumer is one of the following: a breathing system heater of a ventilating apparatus; a breathing gas tube heater; a heater of a transcutaneous measuring cell; or, an infusion pump drive.

7. The arrangement of claim 1, wherein said consumer is a processor in a medical apparatus or in a measuring apparatus; and, said means is adapted to reduce the power inputted by said processor by reducing the clock rate.

8. The arrangement of claim 1, wherein said energy supplied to said consumer is reduced in accordance with operating modes.

9. The arrangement of claim 8, wherein said energy supplied is the full output power when said energy level is 75% in accordance with a first one of said operating modes.

10. The arrangement of claim 8, wherein said energy supplied is reduced by a first pregiven value when said energy level lies between 50% and 75% in accordance with a second one of said operating modes.

11. The arrangement of claim 8, wherein said energy supplied is reduced by a second pregiven value when said energy level lies between 10% and 50% in accordance with a third one of said operating modes.

12. The arrangement of claim 8, wherein said consumer is switched off when said energy level drops below a third pregiven value.

13. A method for operating an arrangement for medical applications which includes a consumer of energy and an energy store for operating the consumer, the energy store having a charge level which changes as energy is drawn therefrom, the method comprising the steps of:
monitoring said charge level of said energy store with a charge level detector; and,
reducing the power output/power input of said consumer when said charge level drops off.
